(19) United States
(12) Patent Application Publication
(10) Pub. No.: US 2022/0054032 A1
(43) Pub. Date: Feb. 24, 2022

(54) METHOD FOR ENHANCING LOCAL EEG SIGNALS AND EEG ELECTRODE DEVICE

(71) Applicant: CHIMEI MEDICAL CENTER, Tainan City (TW)

(72) Inventors: JHI-JOUNG WANG, TAINAN CITY (TW); BOR-SHYH LIN, TAINAN CITY (TW)

(21) Appl. No.: 16/999,412

(22) Filed: Aug. 21, 2020

(51) Int. Cl. A61B 5/04; A61B 5/0478; A61B 5/00

(52) U.S. Cl. CPC A61B 5/04004; A61B 5/0478; A61B 2562/164; A61B 2562/0209; A61B 5/6814

(57) ABSTRACT

A method for enhancing local EEG signals and an EEG electrode device are disclosed. At least one central electrode is provided for receiving a local EEG signal and a plurality of peripheral electrodes arranged around the central electrode are provided for receiving a background EEG signal. The local EEG signal is input to at least one first amplifier of an active dry electrode circuit, and the background EEG signal is input to second amplifiers of the active dry electrode circuit. The amplified local EEG signal is input to a positive terminal of an amplifier of a common-mode EEG signal suppression filter circuit; the amplified background EEG signal is input to a common-mode signal circuit of the common-mode EEG signal suppression filter circuit to generate a common-mode background EEG signal, and the common-mode background EEG signal is input to a negative terminal of the amplifier.

### FIELD OF THE INVENTION

The present invention relates to a method for enhancing local EEG signals and an EEG electrode device, and more particularly to an EEG electrode device having at least one central electrode and peripheral electrodes around the central electrode to obtain local EEG signals and background EEG signals and further to amplify and remove a common-mode background EEG signal generated by the background EEG signals through filtering, so as to enhance the local EEG signals.

### BACKGROUND OF THE INVENTION

EEG is an electrophysiological monitoring method to record electrical activity of the brain of a patient, and it has been widely used in clinical practice. In clinical practice, electrically conductive gel is usually used with conventional Ag/Ag or Au electrodes to measure EEG signals. However, the use of electrically conductive gel for long-term EEG measurement will encounter the drying and hardening problems.

Therefore, many dry electrodes have been developed to improve the above problems. Dry electrodes based on microelectromechanical systems (MEMS) adopt a semi-invasive method, and the manufacturing cost is relatively expensive. Some different electrically conductive materials, such as electrically conductive rubber, fabric, polymer foam, and the like may be applied to dry electrodes. However, the skin-electrode impedance of these electrodes is still higher than that of traditional electrodes using electrically conductive gel. In addition, it is still difficult to use these electrodes to measure EEG signals on the hair area. Unless the hair is separated, these dry electrodes are not in close contact with the skin.

In order to solve the problem that it is difficult to measure EEG signals on the hair area, some comb-shaped dry electrodes are developed on the market. Although these special dry electrodes can increase the contact area between the skin and the electrode on the hair area, the skin-electrode impedance is still much higher than that of traditional electrodes using electrically conductive gel. Besides, the EEG signals are extremely weak and easily interfered by other background EEG signals or physiological electrical signals.

### SUMMARY OF THE INVENTION

In order to improve EEG signals measured by dry electrodes for interpretation, the present invention provides an EEG electrode device for enhancing local EEG signals. The EEG electrode device comprises an EEG signal receiving electrode assembly and an EEG signal enhancement circuit.

The EEG signal receiving electrode assembly includes an electrode substrate, at least one central electrode, and a plurality of peripheral electrodes. The central electrode and the peripheral electrodes are disposed on the electrode substrate. The peripheral electrodes are arranged around the central electrode. The EEG signal enhancement circuit includes an active dry electrode circuit and a common-mode EEG signal suppression filter circuit. The active dry electrode circuit includes at least one first amplifier connected with the central electrode and a plurality of second amplifiers connected with the respective peripheral electrodes. The common-mode EEG signal suppression filter circuit includes an amplifier and a common-mode signal circuit. The first amplifier is connected to a positive terminal of the amplifier. The second amplifiers are connected to the common-mode signal circuit. The common-mode signal circuit is connected to a negative terminal of the amplifier.

Preferably, the common-mode signal circuit is an averaging circuit.

Preferably, the EEG signal receiving electrode assembly is a comb-shaped electrode assembly.

Preferably, the electrode substrate has a circular shape, the central electrode is located on a center of the electrode substrate, and the peripheral electrodes are located on a periphery of the electrode substrate.

Preferably, the central electrode and the peripheral electrodes each include an electrically conductive sleeve, an electrically conductive elastic member, and a pin electrode. The electrically conductive sleeve is welded to the electrode substrate. The electrically conductive elastic member is inserted into the electrically conductive sleeve. One end of the pin electrode is inserted into the electrically conductive sleeve and pressed against the electrically conductive elastic member, and another end of the pin electrode extends out of the electrically conductive sleeve, so that the pin electrode is stretchable relative to the electrically conductive sleeve.

The present invention also provides a method for enhancing local EEG signals, comprising:

providing at least one central electrode for receiving a local EEG signal and a plurality of peripheral electrodes arranged around the central electrode for receiving a background EEG signal; the local EEG signal being input to at least one first amplifier of an active dry electrode circuit; the background EEG signal being input to second amplifiers of the active dry electrode circuit; thereby reducing input signal attenuation and amplifying the local EEG signal and the background EEG signal; the amplified local EEG signal being input to a positive terminal of an amplifier of a common-mode EEG signal suppression filter circuit; the amplified background EEG signal being input to a common-mode signal circuit of the common-mode EEG signal suppression filter circuit to generate a common-mode background EEG signal, the common-mode background EEG signal being input to a negative terminal of the amplifier; thereby removing the common-mode background EEG signal and enhancing the local EEG signal.

Preferably, the common-mode signal circuit is an averaging circuit to generate the common-mode background EEG signal by using an averaging method.

Preferably, the central electrode and the peripheral electrodes are stretchable to fit a patient's head shape.

The following effects can be achieved through the above technical features:

1. The electrodes provided by the present invention are dry electrodes, which can obtain EEG signals with sufficient strength for interpretation without using electrically conductive gel.

2. The EEG signal receiving electrode assembly of the present invention is a comb-shaped dry electrode assembly, and the central electrode and the peripheral electrodes are stretchable to be in close contact with the scalp so as to fit the patient's head shape in a good contact state.

3. The invention utilizes an active dry electrode circuit to amplify local EEG signals and background EEG signals, so as to increase the strength of the received EEG signals and to avoid signal attenuation and phase distortion and to reduce a common-mode rejection ratio. Then, the common-mode signal circuit is used to obtain the common-mode background EEG signal from the background EEG signal to remove the common-mode signal of the local EEG signal and increase the strength of the local EEG signal.

### BRIEF DESCRIPTION OF THE DRAWINGS

FIG. 1 is a schematic view of the EEG electrode device according to an embodiment of the present invention;

FIG. 2 is a perspective view of the EEG signal receiving electrode assembly according to an embodiment of the present invention;

FIG. 3 is a cross-sectional view of the EEG signal receiving electrode assembly according to an embodiment of the present invention;

FIG. 4 is a schematic view of the EEG signal receiving electrode assembly when in use according to an embodiment of the present invention;

FIG. 5 illustrates the electroencephalogram of original steady-state visual evoked potentials without using the EEG signal enhancement circuit according to an embodiment of the present invention;

FIG. 6 illustrates the frequency spectrum of the electroencephalogram of the steady-state visual evoked potentials without using the EEG signal enhancement circuit according to an embodiment of the present invention;

FIG. 7 illustrates the electroencephalogram of original steady-state visual evoked potentials using the EEG signal enhancement circuit according to an embodiment of the present invention; and

FIG. 8 illustrates the frequency spectrum of the electroencephalogram of the steady-state visual evoked potentials using the EEG signal enhancement circuit according to an embodiment of the present invention.

### DETAILED DESCRIPTION OF THE PREFERRED EMBODIMENTS

Embodiments of the present invention will now be described, by way of example only, with reference to the accompanying drawings.

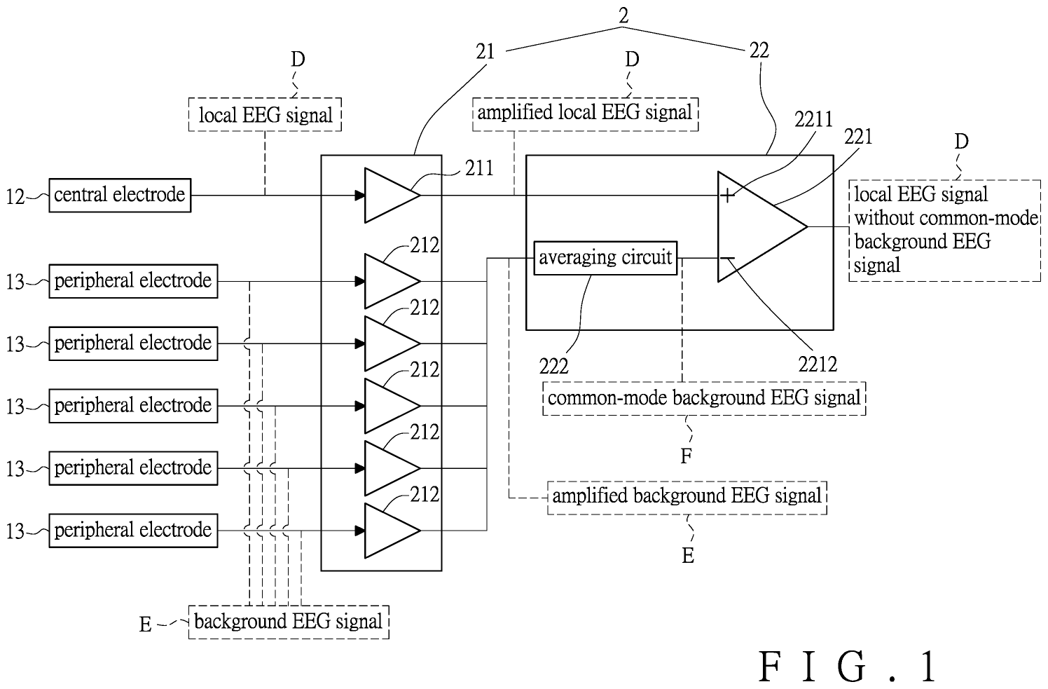

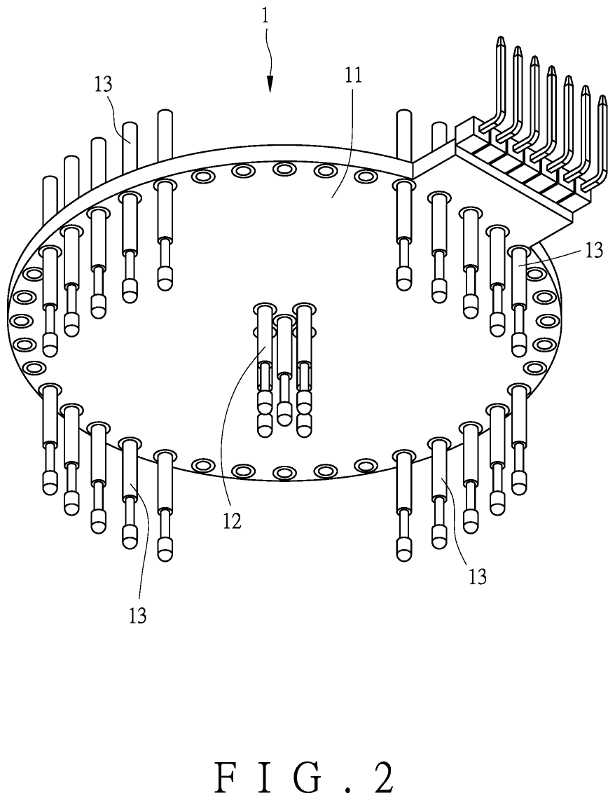

As shown in FIG. 1 and FIG. 2, an EEG electrode device for enhancing local EEG signals according to an embodiment of the present invention comprises an EEG signal receiving electrode assembly 1 and an EEG signal enhancement circuit 2.

The EEG signal receiving electrode assembly 1 includes an electrode substrate 11, at least one central electrode 12, and a plurality of peripheral electrodes 13. The central electrode 12 and the peripheral electrodes 13 are disposed on the electrode substrate 11. The peripheral electrodes 13 are arranged around the central electrode 12. Preferably, the EEG signal receiving electrode assembly 1 is a comb-shaped dry electrode assembly. The electrode substrate 11 has a circular shape. The central electrode 12 is located on the center of the electrode substrate 11, and the peripheral electrodes 13 are located on the periphery of the electrode substrate 11.

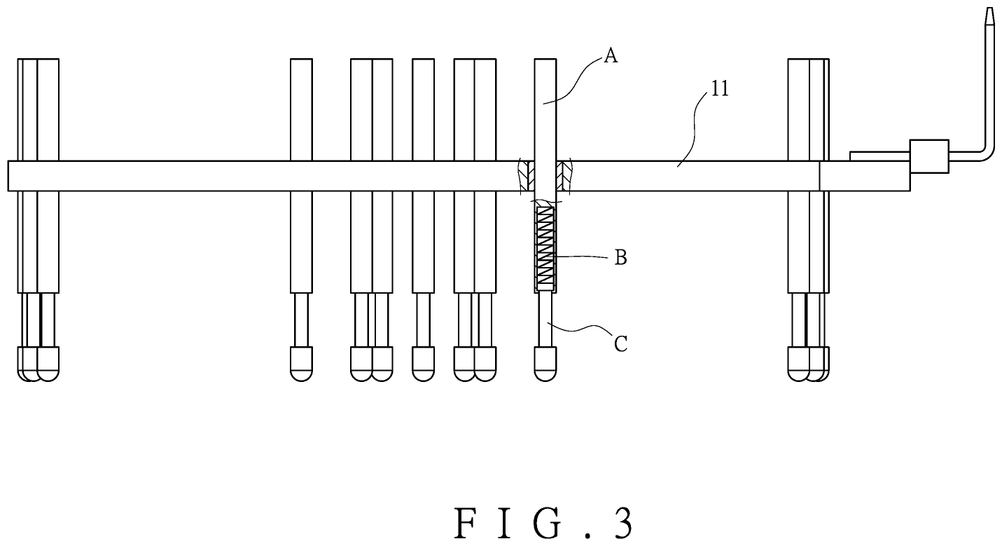

Referring to FIG. 2 and FIG. 3, the central electrode 12 and the peripheral electrodes 13 of this embodiment are stretchable. Specifically, the central electrode 12 and the peripheral electrodes 13 each include an electrically conductive sleeve A, an electrically conductive elastic member B, and a pin electrode C. The electrically conductive sleeve A is welded to the electrode substrate 11. The electrically conductive elastic member B is inserted into the electrically conductive sleeve A. One end of the pin electrode C is inserted into the electrically conductive sleeve A and pressed against the electrically conductive elastic member B, and the other end of the pin electrode C extends out of the electrically conductive sleeve A, so that the pin electrode C is stretchable relative to the electrically conductive sleeve A.

Referring to FIG. 1, the EEG signal enhancement circuit 2 includes an active dry electrode circuit 21 and a common-mode EEG signal suppression filter circuit 22. The active dry electrode circuit 21 includes at least one first amplifier 211 connected with the central electrode 12 and a plurality of second amplifiers 212 connected with the respective peripheral electrodes 13. The common-mode EEG signal suppression filter circuit 22 includes an amplifier 221 and a common-mode signal circuit. The common-mode signal circuit is an averaging circuit 222. The first amplifier 211 is connected to a positive terminal 2211 of the amplifier 221. The second amplifiers 212 are connected to the averaging circuit 222, and the averaging circuit 222 is connected to a negative terminal 2212 of the amplifier 221.

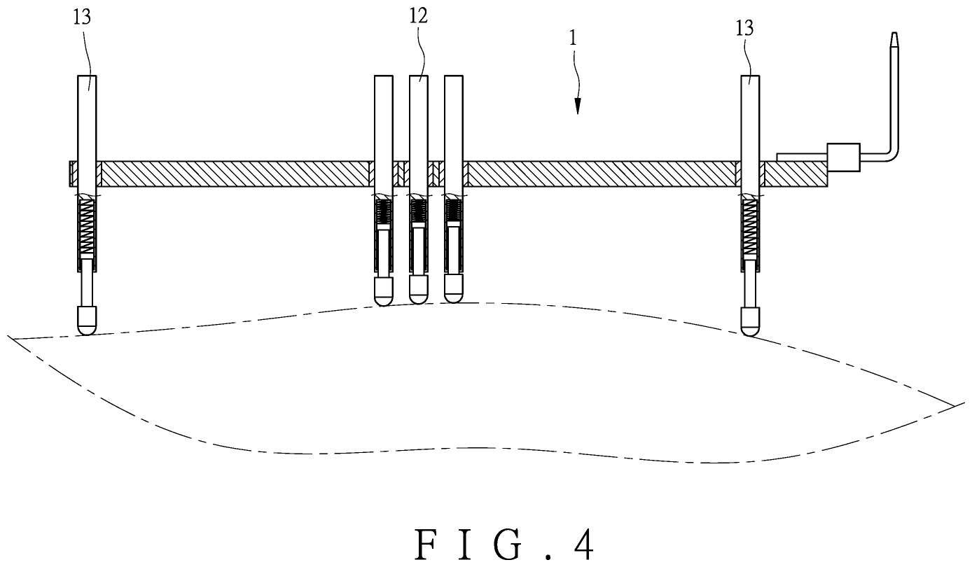

Referring to FIG. 4, for recording electrical activity of the brain of a patient, the electrodes of the EEG signal receiving electrode assembly 1 are placed along the scalp. Because the head is curved, the central electrode 12 and the peripheral electrodes 13 are stretchable so that the central electrode 12 and the peripheral electrodes 13 are in close contact with the scalp of the patient so as to fit the patient's head shape in a good contact state, thereby increasing the strength of the received EEG signals.

Referring to FIG. 1 and FIG. 4, the central electrode 12 is configured to receive a local EEG signal D of the patient, and the peripheral electrodes 13 are configured to receive a background EEG signal E of the patient. The local EEG signal D is input to the first amplifier 211 of the active dry electrode circuit 21; the background EEG signal E is input to the second amplifiers 212 of the active dry electrode circuit 21; thereby reducing the input signal attenuation and amplifying the local EEG signal D and the background EEG signal. The amplified local EEG signal D is input to the positive terminal 2211 of the amplifier 221 of the common-mode EEG signal suppression filter circuit 22; the amplified background EEG signal E is input to the averaging circuit 222 of the common-mode EEG signal suppression filter circuit 22 to generate a common-mode background EEG signal F, and the common-mode background EEG signal F is input to the negative terminal 2212 of the amplifier 221; thereby removing the common-mode background EEG signal F and enhancing the local EEG signal D.

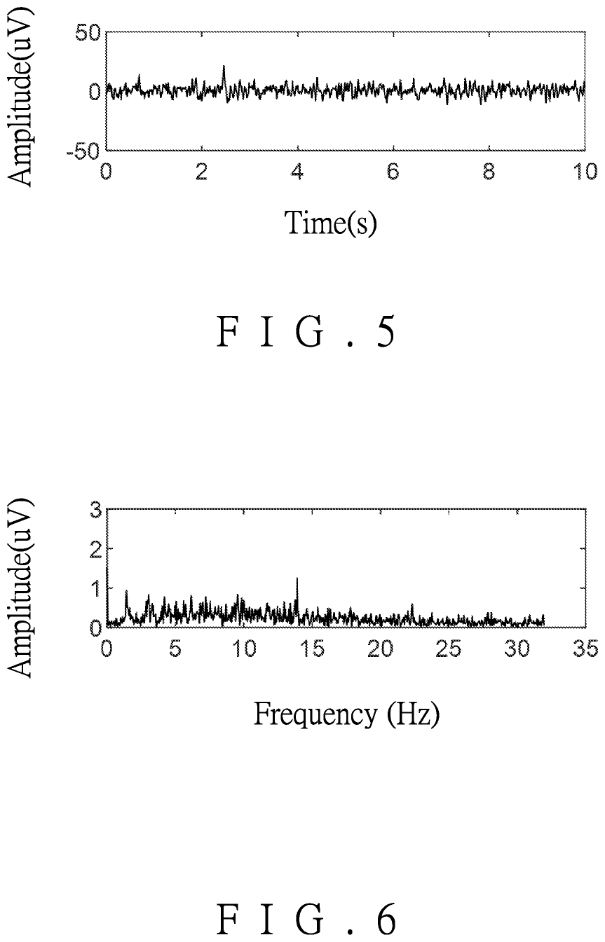

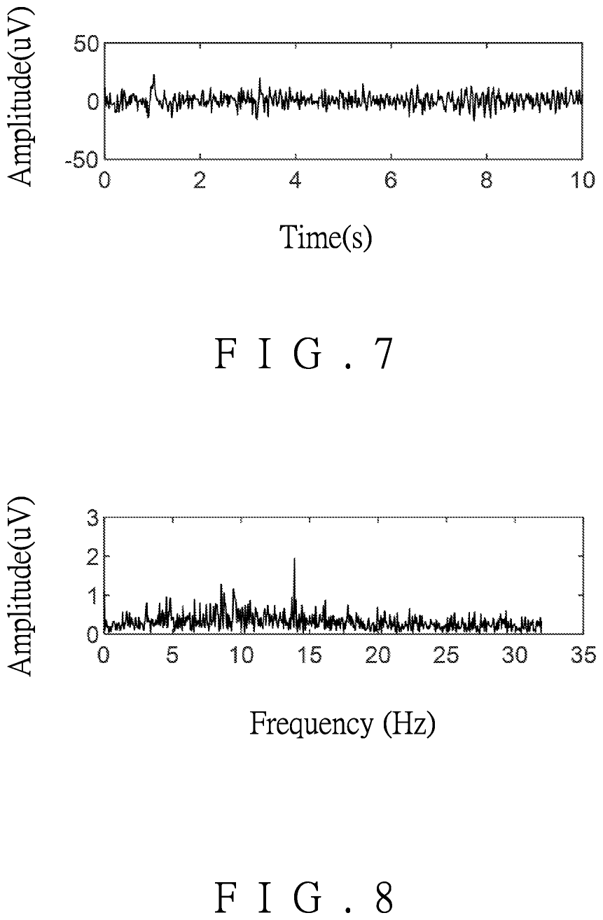

Referring to FIGS. 5-8, the EEG signal receiving electrode assembly 1 is used to measure steady-state visually evoked potentials (SSVEP) response for 14 Hz, and the measurement area is the main visual cortex (Oz). FIG. 5 and FIG. 6 show the original EEG signals and their frequency spectrums of the measured local EEG signals that have not been processed by the EEG signal enhancement circuit 2. FIG. 7 and FIG. 8 show the original EEG signals and their frequency spectrums of the measured local EEG signals processed by the EEG signal enhancement circuit 2. According to the comparison, it is found that the spectral characteristics of the steady-state visual evoked potentials after being processed by the EEG signal enhancement circuit 2 can be enhanced by about 9 dB on average, which overcomes the problem that the skin-electrode impedance is too high so the local EEG signal is weak and that the weak local EEG signal is easily interfered by the background EEG signals or physiological electrical signals.

Although particular embodiments of the present invention have been described in detail for purposes of illustration, various modifications and enhancements may be made without departing from the spirit and scope of the present invention. Accordingly, the present invention is not to be limited except as by the appended claims.

What is claimed is:

1. An EEG electrode device for enhancing local EEG signals, comprising:
an EEG signal receiving electrode assembly, including an electrode substrate, at least one central electrode and a plurality of peripheral electrodes, the central electrode and the peripheral electrodes being disposed on the electrode substrate, the peripheral electrodes being arranged around the central electrode;
an EEG signal enhancement circuit, including an active dry electrode circuit and a common-mode EEG signal suppression filter circuit, the active dry electrode circuit including at least one first amplifier connected with the central electrode and a plurality of second amplifiers connected with the respective peripheral electrodes, the common-mode EEG signal suppression filter circuit including an amplifier and a common-mode signal circuit, the first amplifier being connected to a positive terminal of the amplifier, the second amplifiers being connected to the common-mode signal circuit, the common-mode signal circuit being connected to a negative terminal of the amplifier.

2. The EEG electrode device as claimed in claim 1, wherein the common-mode signal circuit is an averaging circuit.

3. The EEG electrode device as claimed in claim 1, wherein the EEG signal receiving electrode assembly is a comb-shaped electrode assembly.

4. The EEG electrode device as claimed in claim 3, wherein the electrode substrate has a circular shape, the central electrode is located on a center of the electrode substrate, and the peripheral electrodes are located on a periphery of the electrode substrate.

5. The EEG electrode device as claimed in claim 3, wherein the central electrode and the peripheral electrodes each include an electrically conductive sleeve, an electrically conductive elastic member and a pin electrode, the electrically conductive sleeve is welded to the electrode substrate, the electrically conductive elastic member is inserted into the electrically conductive sleeve, one end of the pin electrode is inserted into the electrically conductive sleeve and pressed against the electrically conductive elastic member, and another end of the pin electrode extends out of the electrically conductive sleeve, so that the pin electrode is stretchable relative to the electrically conductive sleeve.

6. A method for enhancing local EEG signals, comprising:
providing at least one central electrode for receiving a local EEG signal and a plurality of peripheral electrodes arranged around the central electrode for receiving a background EEG signal;
the local EEG signal being input to at least one first amplifier of an active dry electrode circuit; the background EEG signal being input to second amplifiers of the active dry electrode circuit; thereby reducing input signal attenuation and amplifying the local EEG signal and the background EEG signal;
the amplified local EEG signal being input to a positive terminal of an amplifier of a common-mode EEG signal suppression filter circuit; the amplified background EEG signal being input to a common-mode signal circuit of the common-mode EEG signal suppression filter circuit to generate a common-mode background EEG signal, the common-mode background EEG signal being input to a negative terminal of the amplifier; thereby removing the common-mode background EEG signal and enhancing the local EEG signal.

7. The method as claimed in claim 6, wherein the common-mode signal circuit is an averaging circuit to generate the common-mode background EEG signal by using an averaging method.

8. The method as claimed in claim 6, wherein the central electrode and the peripheral electrodes are stretchable to fit a patient's head shape.
